(19) United States
(12) Patent Application Publication
(10) Pub. No.: US 2010/0256447 A1
(43) Pub. Date: Oct. 7, 2010

(54) VIRTUAL CHANNEL ENABLING DEVICE FOR USE IN ENDOSCOPIC INSTRUMENT INSERTION AND BODY CAVITY CLEANSING

(76) Inventors: Shay Dubi, Tel Aviv (IL); Eran Hirszowicz, Ramat Gan (IL); David Nitsan, Tel Aviv (IL)

Correspondence Address: NIXON & VANDERHYE, PC, 901 NORTH GLEBE ROAD, 11TH FLOOR, ARLINGTON, VA, 22203, US

(21) Appl. No.: 12/733,855

(22) PCT Filed: Sep. 24, 2008
(86) PCT No.: PCT/IB2008/053881

§ 371 Date: Jun. 7, 2010

Related U.S. Application Data
(60) Provisional application No. 60/974,490, filed on Sep. 24, 2007.

(51) Int. Cl. A61B 1/04; A61M 29/00

(52) U.S. Cl. 600/115; 606/192

(57) ABSTRACT

The present invention is primarily directed to a sleeve device comprising a thin-walled elongate internal tubular membrane which is suitable for being fitted around the external surface of an elongate medical instrument, wherein at least one longitudinally-disposed inflatable channel having a closed distal end is affixed to the outer surface of said membrane, and wherein each of said inflatable channels is capable of being expanded laterally such that an elongate medical instrument fitted with said sleeve and inserted into a body cavity would become anchored within said cavity, and such that the walls of said inflatable channel(s) would define the boundaries of one or more additional longitudinally-disposed large volume channels.

### FIELD OF THE INVENTION

The present invention relates to a device which may be used to cleanse the colon and remove colonic content, and which may also be used to inject medications and/or insert surgical instruments and accessories into any area of the colon or other body cavity. The device of the present invention may thus be used for endo-surgical applications such as endoscopic sub-mucosal dissection (ESD) and natural orifice transluminal endoscopic surgery (NOTES).

### BACKGROUND OF THE INVENTION

Colorectal cancer is the third most common diagnosed cancer in both men and women and the second leading cause of cancer deaths in the U.S.

Colonoscopy is an accepted method for evaluation of the colon and screening for colorectal cancer. The diagnostic accuracy and the therapeutic safety of colonoscopy depend on the quality of the colonic cleansing, or preparation. The ideal preparation for colonoscopy would reliably empty the colon of all fecal material in a rapid fashion without causing damage to the colonic tissues. However, in practice, it is frequently found during colonoscopy that fecal debris is left in place due to inadequate preparation. This is reported to be found in up to 20% of cases, and may cause the termination of the procedure [Toledo T K, DiPalma J A. Review article: colon cleansing preparation for gastrointestinal procedures. Aliment Pharmacol Ther 2001; 15:605-11. Leaper M, Johnston M J, Barclay M, Dobbs B R, Frizelle F A. Reasons for failure to diagnose colorectal carcinoma at colonoscopy. Endoscopy 2004; 36:499-503].

Thus, there would be a significant advantage if a colonic cleansing device were available, which may cleanse the colon during colonoscopy procedures, without requiring termination of the procedure or removal of the colonoscope.

Several potential solutions to this problem have been proposed in the prior art. However, these solutions have generally suffered from one or both of the following disadvantages:
they do not permit the simultaneous use of the endoscope (e.g. for viewing the interior of the body cavity and/or manipulating instrumentation in one or more working channels) together with means for performing irrigation of the colon with a cleaning fluid and aspiration of said fluid;
the channel used for aspirating the fecal debris and/or other solid, semi-solid and liquid matter from the body cavity being cleaned is either incapable of transferring large volumes of such matter—for example the volume of the channel is too small or the walls thereof collapse under the application of high suction pressures—or said channel significantly increases the cross sectional profile of the endoscope to a problematic degree.

Examples of such prior art solutions include the sheath-like devices disclosed in U.S. Pat. No. 5,025,778 and WO 93/11698. The channel in the device in the former publication which may be used for aspiration is of small volume, and requires the insertion of ancillary elements (such as rigidly stiff tubes) in order to prevent collapse under suction. In the device disclosed in WO 93/11698, channels suitable for use in aspiration need to be constructed such that they permanently maintain their tubular form, thereby either increasing the cross section profile of the device or limiting the volume of fecal contents that may be aspirated per unit time.

There is therefore a pressing need for a colonic cleansing device which permits the efficient irrigation and high-volume aspiration of fecal debris during an endoscopic procedure, and which may be used for this purpose without either. removing the endoscope from the body cavity or comprising the functionality of said endoscope in any way.

Many of the technical problems related to development of appropriate endoscopic instrumentation for colonic cleansing are also encountered in relation to devices used in other endoscopic procedures such as NOTES. Thus, for example, on the one hand, there is a need for a device having improved maneuverability and control of the instrumentation that is normally passed through the endoscopic channels in the NOTES procedure. On the other hand, many potential solutions to this problem would necessitate an increase in both cross sectional size of the endoscope and the amount of friction offered thereby during endoscope insertion and manipulation. A need thus exists for a technical solution that would improve instrument manipulation capability without compromising endoscopic advancement.

### SUMMARY OF THE INVENTION

The device of the present invention is a sleeve-like device intended for use in conjunction with medical instruments such as endoscopes, catheters and drain tubes which are normally inserted and operated within body cavities. The primary purpose of the device is to create additional tooling channels associated with the aforesaid medical instruments which may be used for various endoscopic procedures.

One example of a clinical use of the device of the present invention in which the advantages and benefits thereof are readily seen is the cleaning of body cavities during various medical procedures (such as endoscopy). The various favorable structural and functional properties of the device permit irrigation of debris located within the body cavity, and aspiration of same at high pressure without collapse of either the aspiration channel or of the walls of the body cavity. Advantageously, this cleansing function may be performed without preventing or hindering in any way the normal functioning of the medical instrument, thereby obviating the need to cease operation of said instrument or to remove said instrument from the body cavity, when the operator wishes to clean regions of said cavity.

A further highly advantageous feature of the present invention is the fact that the aforementioned aspiration channels are formed only once the device of the present invention has been inserted into the body cavity and immobilized therein. Thus, the insertion and controlled movement of the device within the body cavity is greatly enhanced by the absence of a fixed external large volume aspiration lumen.

A still further highly advantageous feature of the present invention is the ability to use the additional lumens for insertion of endoscopic tools and accessories to facilitate surgical procedures. In particular, the tooling channels of the present device also provide the ability for the operator to control the distance and directional vector of the instruments emerging from said channels. This feature is of particularly benefit in advanced endoscopic procedures such as (but not limited to) ESD and NOTES.

The present invention is thus primarily directed to a sleeve device comprising a thin-walled elongate internal tubular membrane which is suitable for being fitted around the external surface of an elongate medical instrument,
wherein at least one longitudinally-disposed inflatable channel having a closed distal end is affixed to the outer surface of said membrane,
and wherein each of said inflatable channels is capable of being expanded laterally such that an elongate medical instrument fitted with said sleeve and inserted into a body cavity would become anchored within said cavity, and such that the walls of said inflatable channel(s) would define the boundaries of one or more additional longitudinally-disposed large volume channels.

The term “large volume” channels refers to the fact that the additional longitudinally-disposed “virtual channels” (which are passively created as a consequence of expansion of the inflatable channels and anchoring of the device) may have cross-sectional areas and hence volumes far greater than commonly encountered in endoscopic instruments. In general, each of these channels may have a volume in the range of 5-200 ml. These figures are, however, given for the sake of illustration only; the actual volume of the virtual channels may deviate to either side of this exemplary range.

For the sake of clarity, it is to be noted that in the context of the present disclosure, “distal” refers to the direction away from the operator, such that the distal end of the device is the leading end, i.e. the end that is first inserted into the body cavity. “Proximal”, therefore, refers to the opposite (trailing) end, i.e. the end of the device which is closer to the operator.

One particularly advantageous feature of the device of the present invention is the fact that the large volume “virtual” channels created upon inflation within a body cavity are capable of substantially retaining their initial volume and cross-sectional area upon the application of very high suction pressures. Thus in one preferred embodiment of the present invention, the virtual channels are capable of withstanding the application of suction pressures of up to −760 mmHg (−1 atm) to their proximal ends without any substantial deformation. The terms “substantially retaining volume and cross-sectional area” and “without substantial deformation” are intended to convey the mean that at the indicated high suction pressure, the “virtual channels” retain most of their maximal capability for aspirating solid and liquid materials.

In one preferred embodiment, the device further comprises one or more open-ended longitudinally disposed non-inflatable channels affixed to the aforementioned internal tubular membrane, wherein said channels are capable of allowing the passage of fluids along their length.

In a further highly preferred embodiment, the device of the present invention further comprises an elongate longitudinally-disposed external tubular membrane which encloses all of the other elements of said device within its lumen.

As mentioned previously, the device of the present invention may be used in conjunction with any suitable elongate medical instrument that is normally inserted and operated within a body cavity or passage. However, in one preferred embodiment, the internal tubular membrane of said device is suitable (in terms of dimensions and form) for being fitted around the external surface of an endoscope, such as a colonoscope, enteroscope, sigmoidoscope or bronchoscope.

In order to permit connection of the device with external sources of suction pressure, balloon inflation fluid (such as saline or water) and cleaning fluid, the proximal end thereof may comprise means for connecting the open proximal ends of the various channels with said suction pressure and fluid supply sources.

In one preferred embodiment, each of the inflatable channels is constructed as a single balloon-lie element, having a closed distal end and an open proximal end.

In an alternative preferred embodiment, however, each inflatable channel comprises a chain of separate segments, wherein each segment is in fluid communication with its neighbors by means of a narrow opening. In an alternative version of this embodiment of the device, each inflatable channel may comprise a plurality of separate segments, wherein each segment is isolated from its neighbor and may be independently inflated and deflated by means of a separate inflation/deflation line attached thereto.

In another preferred embodiment, the device may also comprise an expandable balloon located at the distal end thereof, such that upon inflation thereof, balloon blocks the distal openings of the various channels. This distal balloon (optionally together with the circumferential balloons that are disclosed immediately hereinbelow), may be used to assist in the clearance of solid and semi-solid material that has become trapped within the large-volume “virtual channels”.

The device may also further comprise one or more circumferentially located balloons each of which is affixed to the external surface of the device in an annular manner, such that upon inflation thereof, the contents of the large volume channels are compressed. The co-coordinated inflation of a series of such balloons may be used to generate a proximally-directed peristaltic-like wave of pressure in the region of the trapped debris, thereby encouraging the removal thereof from the “virtual channel”.

In one preferred embodiment, the device of the present invention further comprises one or more auxiliary balloons attached to the outer surface of the internal membrane close to the distal end of said device. These balloons (which will be described in more detail hereinbelow) may be either continuous, ring-like balloons, or alternatively, may be individual, discrete balloons located in proximity to the positions where the “virtual channels” will form upon expansion of the inflatable channels.

All of the aforementioned inflatable channels and auxiliary balloons are connected by means of supply lines or channels to a reservoir of inflation fluid (such as saline) which resides proximal to the device, outside of the patient's body.

In another embodiment, the device may further comprise a proximal plug fitted in an annular manner around the external surface of said device, wherein said plug is adapted to form a fluid-tight seal when placed in the anal opening.

In yet another preferred embodiment of the invention, the device may further comprise pressure sensor elements located in various regions of said device, wherein said sensor elements are capable of detecting and reporting the fluid pressure levels within said regions.

In another aspect, the present invention also provides a system for use in cleansing a body cavity comprising:
a device according to any one of the embodiments disclosed hereinabove and described in more detail hereinbelow; and
a proximal control console.

In addition, the above-defined system may also comprise:
an extracorporeal pumping device capable of causing flow of cleaning fluids through one or more lumens of said device; and
an extracorporeal suction pressure source capable of causing aspiration of fluid, solid and semi-solid material through the large volume lumens of said device;
a separate source of fluid for expanding the inflatable channels and/or other expandable elements of the device and a pumping device for delivering said fluid to said inflatable channels and/or other expandable elements.

In a further embodiment, the aforementioned proximal control console of the presently-disclosed system comprises:
a display unit;
means for controlling one or more pumping devices;
means for controlling a suction pressure source; and
appropriate software for operating and co-coordinating the passage of cleaning fluid and expansion fluid into the device, and the application of suction pressure thereto.

In another aspect, the present invention also provides a method for cleansing a body cavity comprising the steps of:
a) fitting a sleeve device around the external surface of an elongate medical instrument, wherein said device comprises a thin-walled elongate internal tubular membrane to the outer surface of which is fixed one or more inflatable channels, and optionally comprises one or more non-inflatable fluid-supply channels;
b) insertion of said elongate medical instrument together with the fitted sleeve into a body cavity, and advancement of said instrument until the distal end thereof is situated in the desired region;
c) inflation of said inflatable channels, such that said medical instrument becomes anchored within the desired region of the body cavity, and such that longitudinally-disposed large-volume virtual channels are created between said inflatable channels;
d) introduction of cleaning fluid into either the large-volume virtual channels or the non-inflatable fluid-supply channels, thereby causing the creating of fluid spray jets through openings located in either said virtual channels or in said fluid-supply channels, such that said fluid spray jets are directed outwardly towards debris located in said body cavity; and
e) performing suction of fluid, solid and semi-solid debris and other material from the body cavity;
f) deflation of said inflatable channels; and
g) optionally, movement of said medical instrument to another location within the body cavity to be cleansed or treated;
wherein steps (b) to (g) do not adversely affect the ability of the operator to operate said medical instrument in the normal manner.

In one preferred embodiment of the method of the present invention, the body cavity to be cleansed is the colon, and the elongate medical instrument is a colonoscope.

The present invention is also directed to a method for delivering endoscopic tools and accessories to a desired surgical site and for controlling the location of said tools and accessories at said site, comprising the steps of:
a) fitting a sleeve device around the external surface of an elongate medical instrument, wherein said device comprises a thin-walled elongate internal tubular membrane to the outer surface of which is fixed one or more inflatable channels, and optionally comprises one or more auxiliary balloons located on the outer surface of said membrane proximate to the distal end of said membrane;
b) insertion of said elongate medical instrument together with the fitted sleeve into a body cavity, and advancement of said instrument until the distal end thereof is situated close to the desired operating site;
c) inflation of said inflatable channels, such that said medical instrument becomes anchored within the desired region of the body cavity, and such that longitudinally-disposed large-volume virtual channels are created between said inflatable channels;
d) insertion of said endoscopic tools and/or accessories into one or more of said longitudinally-disposed large-volume virtual channels, and the distal advancement of said tools and accessories through said channels until said tools become located at the desired operating site; and
e) optionally inflating one or more of said auxiliary balloons in order to control the angle of exit of one or more of said tools from said virtual channels and/or the separation distance between multiple tools;
wherein steps (d) and (e) may be performed either simultaneously or sequentially, in either order.

In one preferred embodiment, the method described immediately hereinabove is used to control the insertion and positioning of endoscopic surgery tools and accessories as part of a natural orifice transluminal endoscopic surgery (NOTES) procedure.

All the above and other characteristics and advantages of the present invention will be further understood from the following illustrative and non-limitative examples of preferred embodiments thereof.

### BRIEF DESCRIPTION OF THE DRAWINGS

The present invention is illustrated by way of example in the figures of the accompanying drawings, in which like references indicate similar elements and in which:

FIG. 1 schematically illustrates an exemplary embodiment of the invention, fitted over a colonoscope, in a deflated state;

FIG. 2 schematically illustrates an exemplary cross section of an exemplary embodiment of the invention, fitted over a colonoscope in a deflated state, within a lumen;

FIG. 3 schematically illustrates an exemplary embodiment of the invention, fitted over a colonoscope, in an inflated state;

FIG. 4 schematically illustrates an exemplary cross section of an exemplary embodiment of the invention, fitted over a colonoscope in an inflated state, within a lumen;

FIG. 5 schematically illustrates the distal head of an exemplary embodiment of the invention, fitted over a colonoscope in an inflated state;

FIG. 6 schematically illustrates the proximal tail of an exemplary embodiment of the invention, fitted over a colonoscope;

FIG. 7 schematically illustrates one embodiment of the distal head of the device fitted with multiple exemplary fluid nozzles;

FIG. 8 schematically illustrates the generation of turbulent flow in a conduit upon rapid change in lumen diameter;

FIG. 9 is a schematic view of a device of the present invention fitted with three longitudinally-disposed inflatable channels, each consisting of a series of separate (but interconnected) inflatable compartments;

FIG. 10 illustrates the use of circumferential balloons in order to apply a retrograde (distal to proximal) series of pressure pulses onto the device of the present invention, in order to assist in removing debris trapped within the virtual lumen(s);

FIG. 11A illustrates the external features of a preferred embodiment of the device of the present invention;

FIG. 11B is a line drawing showing the various component parts of the device illustrated in FIG. 11A;

FIG. 12 is a schematic cross sectional view of a device of the present invention comprising three different longitudinally-disposed inflatable channels (balloons) of different shapes and sizes;

FIG. 13 schematically depicts a longitudinal section of a device of the invention comprising a ring-shaped auxiliary balloon situated in apposition to the distal ends of two virtual channels, and illustrates the manner in which inflation of said balloons may be used to alter the inter-tool separation distance;

FIG. 14 schematically depicts a device in which a ring-shaped auxiliary balloon is located at a more proximal point to that of the device shown in FIG. 13, and in which the inflation of said balloon may be used to alter the exit angle of the surgical tools placed within said channels;

FIG. 15 schematically depicts a device similar to that shown in FIG. 14, but which differs therefrom in its use of two separate auxiliary balloons, which, when one is independently inflated, permits the meeting point of the two tools to be shifted away from the center point of the device.

### DETAILED DESCRIPTION OF PREFERRED EMBODIMENTS

There is, therefore, provided according to the present invention, a novel type of device, constructed in the form of a thin sleeve having various channels attached thereto or formed therein, wherein said device is connected at its proximal end to a specialized pump which can pump fluid (and/or gas) forward within a lumen or lumens of the device, and pump fluid (and/or gas) backward through either a different lumen or the same lumen, as will be further explained in the text. The anterograde pumping action supplies fluid that functions as a fecal dematerialization and degradation agent, allowing easier removal of fecal material, and filling for the anchoring balloon elements of the device, as will be further explained in the text. The device, in its non-active (deflated) state, is fitted over the external surface of a colonoscope (or any other medical instrument or device of elongate form that may be inserted into a body cavity, such as a catheter, intubation tube or drain), in a sleeve-like fashion, prior to performing the colonoscopy procedure. The colonoscope can then be advanced within the colon as regularly performed to conduct the procedure. During the procedure, if the operator encounters debris (fecal material), the device of the invention can be operated to safely and effectively remove the debris, thus cleansing the colon and allowing continuation of the procedure.

Operation of the device may include the steps of:
1. Inflation of anchoring balloon elements.
2. Spraying fluid jets onto the site of the debris.
3. Performing suction of fluid and solid debris from the site.
   (It is to be noted that steps 2 and 3 may be performed sequentially or simultaneously, and may be performed continuously or non-continuously, in a pulsating manner. So that fluid jets to cleanse the fecal material may be continuous or pulsatile, and suction of debris may be continuous or Pulsatile).
4. Deflating anchoring balloons.
5. Continuing the endoscopic procedure in a regular manner.

The same cleansing device and method may be used repeatedly throughout the procedure, if required, without the need for removing the colonoscope from the colon.

In one preferred embodiment, the device comprises two separate sets of channels: firstly, inflatable, balloon-like channels whose functions includes anchoring of the device within the colon) or other body cavity, prevention of intestinal wall collapse during high pressure aspiration and the creation of a series of “virtual channels” (used for high pressure aspiration) upon inflation of said inflatable channels. The second type of channel is a fluid channel, the purpose of which is to carry irrigation fluid pumped from a proximally located extracorporeal reservoir towards a series of jet-spray apertures that perforate said channels at various locations.

In another preferred embodiment, the device comprises a single set of channels, the aforementioned inflatable channels. In this case, the aforementioned virtual channels will be used for both supply of irrigation fluid in one direction, and high-pressure aspiration in the opposite direction.

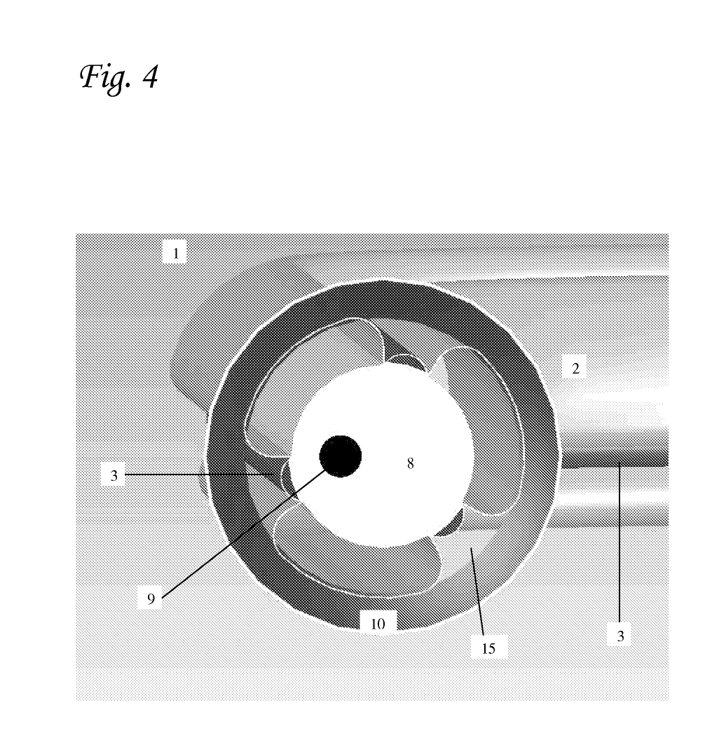

An additional preferred embodiment may include anchoring of the device using the fluid channels (shown as 3 in FIG. 4), thus saving more space for the fluid channels (shown as 3 in FIG. 4) and/or for the “virtual” channel (shown as 15 in FIG. 4).

All of these various embodiments will be described in more detail hereinbelow.

The presently-disclosed device possesses the following desirable and advantageous properties:
Non-disruptive of colonoscope function during the procedure.
Capable of spraying fluid jets of a sufficient pressure and flow intensity to allow immobilization and removal of fluid, semi-fluid and rigid debris within the colon.
Permits viewing of the debris site with the colonoscope during the cleansing procedure.
Ensures good localization of the colonoscope during the cleansing procedure.
Permits aspiration and removal of the fecal debris, with all fluid, semi-fluid, solid elements, for complete colonic cleansing.
Prevents collapse of colonic walls during suction of the debris.
Ensures safety of the procedure, by means of controlling the pressure within the colonic lumen, in order that perforation of the colon will be prevented.
Simplicity of use, thereby increasing operator compliance.
Permits cleansing of the colon with a process that is much more comfortable for the patient than current regimes. Thus, a simple diet without the help of the current laxatives agents, such as Golytely, Halflytely, or alternatively, with lower doses of such agents may be used in conjunction with the device and method of the present invention, thereby increasing patient compliance.
Permits removal of hard feces attached to the colon wall without damaging the colon wall, which is difficult in many cases where the hard feces covers either a polyp and/or suspicious lesion inside colon cavities.
Permits insertion of multiple endoscopic devices
Permits control of the distance between the exit port of the device as well as the vector direction of the devices.
Permits performance of surgical procedures and simultaneous irrigation/aspiration of debris and blood through the virtual lumen of the device of the invention or through the endoscope.
Permits retrieval and extraction of large elements such as surgical tools resected polyps and gall bladder stones.
Permits insertion of surgical devices and accessory items such as gases, pads, antibiotic paste etc.

The above advantages of the present invention are, to a certain degree, a consequence of the unique pumping mechanism employed, which provides the capability for forward and backward pumping, and preferably includes a pressure sensor. Furthermore, the device itself can be deployed on the external surface of a colonoscope or other elongate medical instrument (such as a catheter tube), in a sleeve-like manner. Preferably, the device of the present invention is disposable, and intended for single use only.

The device comprises may comprise some or all of the following elements:
Channels for fluid propagation and/or tooling insertion, the walls of which are perforated at multiple sites to allow the formation of fluid jets.
Closed channels, which are inflated to form “balloon-like” anchoring elements.
A proximal plug-like element, which is inserted into the anus and prevents leakage of fluid and debris during the cleansing procedure. (This element may be freely movable over the disposable element, or, in a different embodiment, may be a separate element.)

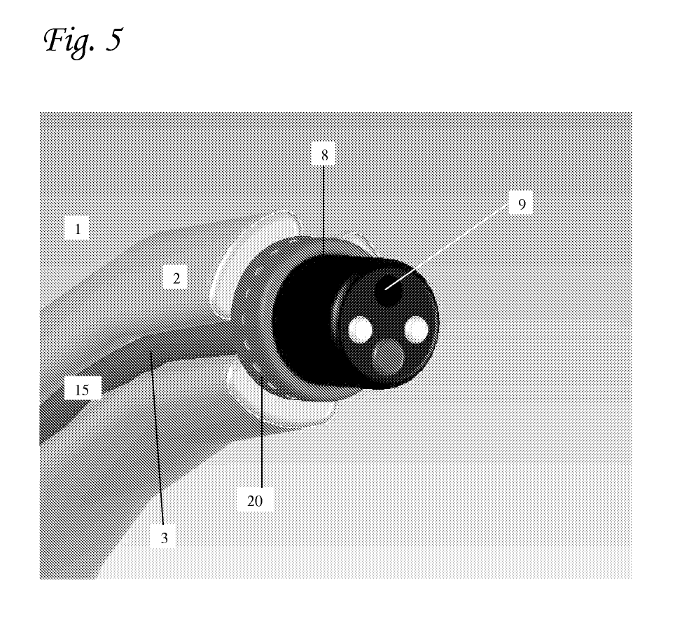

The various advantageous properties of the device that are listed hereinabove are a consequence of its unique structural and functional properties, as explained in the following section:
Non-disruptive of colonoscope function during the procedure: The device is fitted over the colonoscope (like a sleeve) in a deflated state, thus increasing the colonoscope diameter by only 0.1-2 mm, and is elastic or flexible and thus does not interfere with movement of the colonoscope.
Capable of spraying fluid jets of a sufficient pressure and flow intensity to allow immobilization and removal of fluid, semi-fluid and rigid debris within the colon: The fluid channels are external to the colonoscope, thus allowing significantly larger flow compared to the internal colonoscope “working channel”
Permits viewing of the debris site with the colonoscope during the cleansing procedure: During the cleansing process the colonoscope is anchored by the anchoring balloons, and therefore permits continuous viewing of the cleansing procedure.
Ensures good localization of the colonoscope during the cleansing procedure: The anchoring balloons prevent displacement of the colonoscope during the cleansing procedure.
Permits aspiration and removal of the fecal debris, with all fluid, semi-fluid, solid elements, for complete colonic cleansing: The suction channels are formed between every two anchoring balloon elements and the colonic wall (as shown in FIGS. 4 and 5). Due to the difference between the diameter of the colon and the diameter of the colonoscope, these channels are of a wide diameter (approximately 5-15 mm), thus permitting very high flow and suction rates.
Prevents collapse of colonic walls during suction of the debris: The anchoring balloons, when inflated, prevent collapse of the colonic wall during suction. In another preferred embodiment of the device of the invention, there is a membrane between the anchoring balloons, on the lateral surface, this membrane essentially forming a “roof” for every suction channel, thus preventing colon wall collapse into the channel during suction.
Ensures safety of the procedure, by means of controlling the pressure within the colonic lumen, in order that perforation of the colon will be prevented: The pump may include a pressure control element, monitoring fluid pressure, and preventing pressure from crossing a predetermined level. In another preferred embodiment of the device of the invention, the disposable sleeve element includes one or more pressure sensors, to allow monitoring of the intra-colonic luminal pressure during the procedure. In another preferred embodiment the pumping action may be either done by manually adjusting the flow rate and/or using predefined programmed flow rate levels and/or automated flow adjustment using flow controller that are based on sensor elements.
Increase the overall efficacy of the procedure, since it will enable a cleaner colon thus better diagnostic yield.
Increase procedure safety, since it will remove obstructions which are currently irremovable without the need for the physician to exert significant force and pressure in order to overcome such obstructions.
One further advantage of the inflation of the device is that the colon expands and some of the colon wall cavities will “disappear” enabling easier and more effective cleaning.
Permits the performance of complex endosurgical techniques such as NOTES, as a consequence of the ability to determine and accurately control the positioning of multiple instruments beyond the distal end of the endoscope.

In summary, the device of the invention permits effective and safe cleansing of the colon during a colonoscopy procedure, without requiring removal of the colonoscope from the patient's body, and without requiring the use of the internal working channel of the colonoscope, thus preventing the need to prematurely stop the procedure in the case that bowel preparation proves to be insufficiently thorough, and the need to send the patient to perform the procedure again. Thus, the device may be used in the preparation of patients for all lower GI track procedures for which a thoroughly clean colon is a prerequisite.

In addition, the device of the invention allows the monitoring of pressures during a colonoscopy procedure, thus increasing the safety of the procedure, and preventing the occurrence of bowel perforation.

It is to be noted that while the description of the present invention hereinabove and hereinbelow generally refers to the use of the device of the invention within the colonic lumen, this is for illustrative purposes only, and the scope of the invention is intended to encompass a broader use and application of such a device within any lumen of the body, as will be appreciated by the skilled artisan in this field. Other, non-limiting, examples, which will not be further elaborated in the text, are the use with endoscopes in the upper GI system (for example the esophagus, stomach and small intestines), and in the urinary system (for example, in the urinary bladder and ureters).

The present invention is primarily directed to a system, method and apparatus for cleansing the colon during a colonoscopy procedure, without the need for removal of the colonoscope from its operating position or from the body.

In one preferred embodiment of the invention the system includes an extracorporeal pumping unit, with the ability to both deliver fluid into the lumen and extract material (fluid, solid and semi-solid) from the lumen, and a disposable sleeve unit, which is fitted over the colonoscope prior to the procedure, in a deflated state, and is used to immobilize and remove debris when needed, by inflating one or more anchoring balloon elements, spraying fluid jets on the site of the debris, and employing suction to remove the debris.

In some preferred embodiments, the aforementioned pumping unit may be connected to, or form part of, an external control unit comprising a console fitted with a display unit (e.g. an LCD unit) as well as elements for controlling the inflation and deflation of the inflatable balloon-like channels (which will be discussed in more detail hereinbelow). These control elements will also be used, in some embodiments, to control the peristaltic-like inflation and deflation of segmented and multi-chamber inflatable channels, as well as the similar inflation-deflation cycle of circumferential balloons used to dislodge trapped debris and move same in a proximal direction. All of these inflatable elements will be described in more detail hereinbelow. In some embodiments, the external control unit will also receive input from one or more pressure sensors located at different sites within the sleeve-like device. In other preferred embodiments, the external control unit will also receive input from cameras and other detection instruments, positioned within the sleeve-like device.

The fluid jets can assist with the breakdown and shredding of material within the lumen. The device may then extract the material from the colonic lumen, thus cleansing the lumen. Alternatively or additionally, the device may also be used to collect samples or other objects from the lumen such as large resected polyps, gall bladder stones and other foreign objects. In addition, the device can administer specific material, such as medications, into the lumen.

In a preferred embodiment of the invention the lumen is the colonic (large bowel) lumen, and the device is used to cleanse the lumen during a colonoscopy procedure, and/or to collect material from the colon, and/or to deliver medication into the colon.

The fluid jets applied by the device may be generated using any type of appropriate fluid, including, but not limited to, water, saline, cathartic agents, polyethylene glycol, phosphate preparations and medications. The fluid may include small rigid particles to create a mechanical sand-wash effect.

The fluid may be heated within the pump, to various temperatures prior to its expulsion into the colon, for example it may be warmed to about 36 degrees Celsius, thereby accommodating body heat and increasing the comfort of the patient being treated.

As mentioned hereinabove, the fluid that is sprayed into the colon or other body cavity may contain therapeutic agents such as chemotherapy agents (for example Xeloda, Oxaliplatin and CPT) and biologic solutions (for example Erbitux and Avastin).

Suitable pumps that may be used for the purposes of expanding the inflatable channels and for pumping the cleansing fluid include (but are not limited to) simple water pumps, centrifugal, and peristaltic pumps. Similarly, suction pressure may be generated with water pumps and vacuum pumps, as are well know to the skilled artisan in the field.

Exemplary materials for construction of the device of the invention are plastic, elastomers and polymers such as silicon, polyurethane, nylon, Pebax and blends of nylon and Pebax. The device may include elastic and non elastic materials.

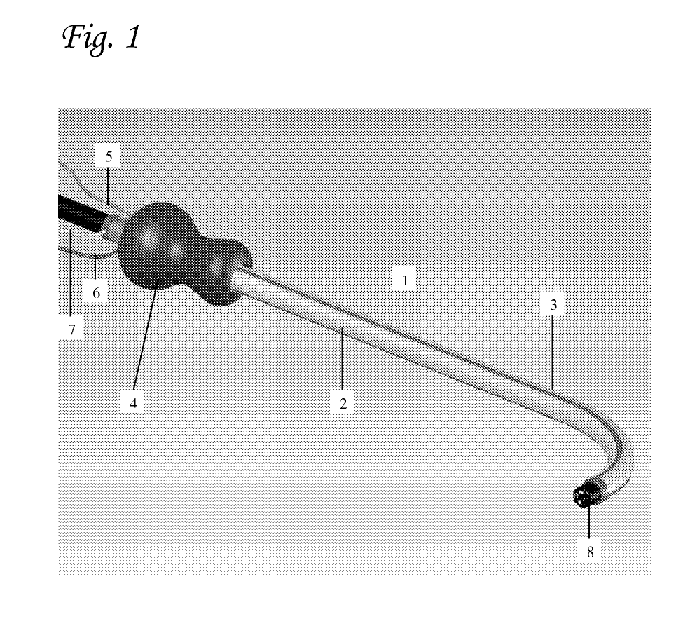

FIG. 1 is a schematic illustration of an exemplary embodiment of the device 1 of the invention, fitted over a colonoscope, in a deflated state. The device includes inflatable anchoring balloon channels 2, which are closed at their distal side. When said channels are filled with gas or fluid, they expand laterally in a balloon-like manner and may thereby be used to anchor the device within the lumen of the colon or other body cavity. The device further comprises fluid channels 3, which have apertures that allow jets of fluid to be expelled into the lumen of the colon.

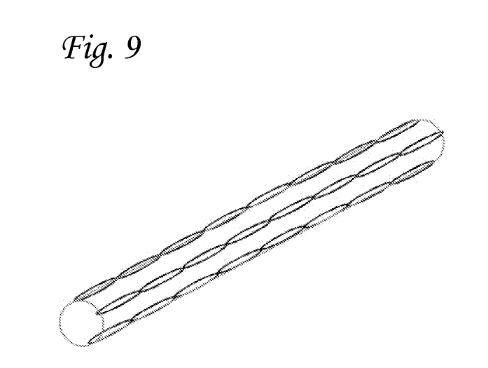

Each of the inflatable anchoring channels 2 may be formed as a single tube, or alternatively may be formed as multiple-chambered tube elements. In one preferred embodiment, this may be achieved by means of constructing the inflatable channel as a series of interconnected chambers (the appearance of which may be likened to a “string of sausages”) which are in fluid connection with each other and with a single inflation/deflation line. This embodiment is schematically illustrated in FIG. 9, which shows three inflatable channels, each consisting of eight separate (but interconnected) inflatable chambers. In a further preferred embodiment, the inflatable channel is constructed as a series of interconnected chambers in which each chamber is (from the point of view of fluid transfer) isolated from each other, each of said chambers being connected to its own, separate, inflation line or channel. One advantage of the use of multiple-chamber inflation channels is that such a construction may increase the lateral force that the channel is able to employ, thus increasing its ability to prevent colon wall collapse during suction of material from the colon. An additional advantage of multiple chamber design is that in the event that one chamber is punctured, the structural integrity of the channel may remain largely intact, thereby preventing its unwanted deflation. A further advantage of such a structure is that its inflation may be controlled such that a peristaltic-like sequence of inflated-deflated chambers may be achieved. This type of inflation sequence is particularly useful when maneuvering the device of the invention around sharp bends and convolutions in the intestinal tract. In particular, the segmented design will prevent balloon kinking which may otherwise sometimes occur during the insertion of single balloon versions, particularly when encountering resistance upon attempting to advance the device around a bend or corner. Similarly, synchronous inflation of a segmented inflation channel which may cause a snake-like movement of said channel (and hence of the device as a whole) may be useful in assisting the initial insertion of the device into the intestinal tract.

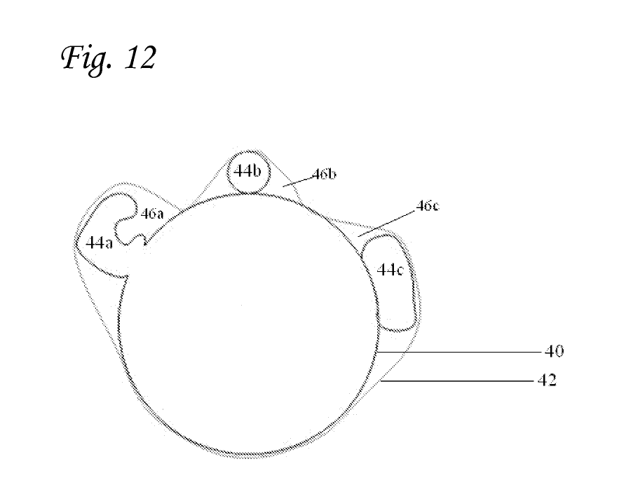

It is to be noted that the inflatable channel balloons need not be restricted to balloons that have a circular cross-sectional shape. Rather, in certain circumstance it will be found advantageous to utilize pre-shaped balloons that may adopt particular conformations upon inflation, thereby altering the size and shape of the virtual channels formed upon said inflation. The effect of incorporating balloons having different expanded shapes on the form and size of the virtual channels is illustrated in FIG. 12, which is a schematic cross sectional view of a device of the present invention comprising three different longitudinally-disposed inflatable channels (balloons) of different shapes and sizes (44a, 44b and 44c.) attached to the inner membrane 40. As will be seen, the corresponding virtual channels (46a, 46b and 46c) which are formed between said shaped inflatable channels and the external membrane 42 each possess a cross sectional shape and area which is a consequence of the particular form and size of the corresponding inflatable channel in its expanded state. It may also be seen from this figure that the use of shaped balloons influences the overall outline shape of the device when inflated (i.e. the envelope contour of the outer membrane 42), as well as determining the size and shape of each individual virtual channel.

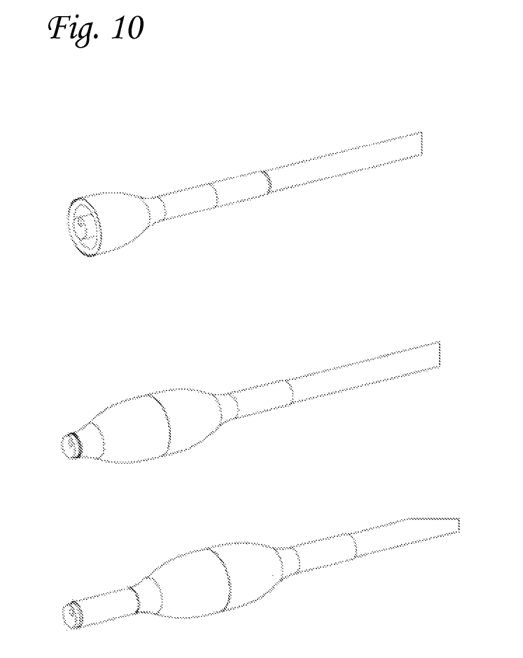

In some preferred embodiments of the present invention, the device further comprises additional inflatable, balloon-like, elements that may be used to assist the retrograde movement (i.e. towards the proximal end of the device) of fecal debris that may sometimes become trapped inside the virtual lumens. Thru, in one embodiment, a balloon situated at the distal extremity of the device is inflated, in order to increase pressure within the virtual lumen(s). Then, a series of two or more circumferential balloons mounted on the external membrane or sleeve of the device are inflated sequentially, thereby applying a constrictive force to the virtual lumen(s) in a peristaltic-like manner, beginning at the distal end of the device and moving proximally. In this way, the trapped solid and/or semi-solid debris within the virtual lumen is both compressed in a radial direction and in a longitudinal (distal-to-proximal) direction, thereby assisting in the transportation of said debris towards the proximal end of the device. A typical example of the use of three such balloons is depicted in FIG. 10, which illustrates (from top to bottom) the inflation sequence, the distal end of the device being on the left side of the figure, and the proximal (anal) end being on the right side.

In other preferred embodiments of the present invention, additional balloons may be incorporated into the device, by means of attaching them to the inner membrane thereof, such that they are positioned adjacent to the median wall of virtual channels. Such balloons may either be short, individual balloons extending over only a small part of the circumference of the inner membrane (i.e. only in those areas above which a virtual channel will appear during use of the device), or alternatively, the balloon may be in the form of a ring, attached around the entire circumference of the inner membrane. Such balloons may be used to selectively apply pressure to the distal portions of one or more virtual channels, in order to change the distance between the distal exits of said channels and hence the distance between the surgical tools and accessories (e.g. needles, graspers, knives, cameras, fiber optics etc.) that are placed in said channels. This ability to control the inter-tool separation distance is of great clinical significance, particularly in complex procedures such as NOTES, and constitutes a highly significant over prior art solutions that are characterized by multiple fixed working channels.

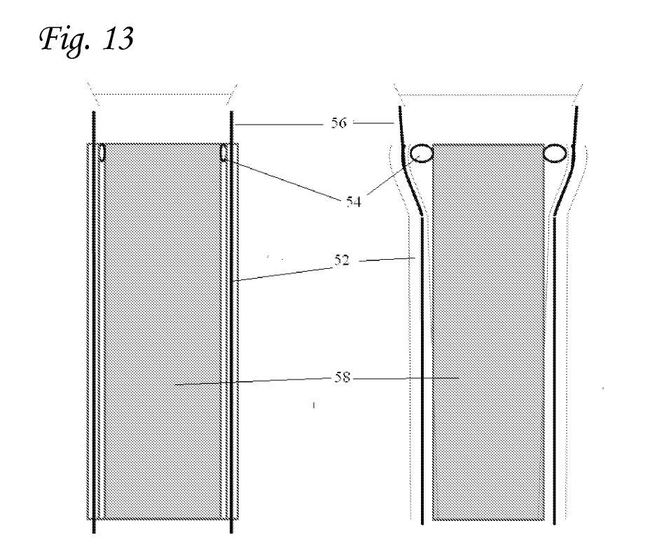

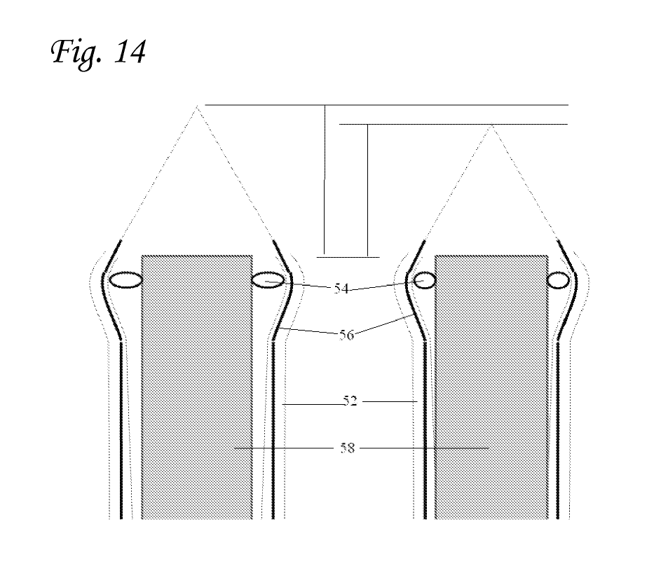

FIG. 13 schematically depicts a longitudinal section of a device of the invention fitted around an endoscope 58. A ring shaped balloon 54, which is located on the medial side of a virtual channel 52, at the distal exit of said channel, is shown in its deflated state in the left side of the figure, and in its inflated state in the right side of the figure. It will be readily observed that the separation distance between two tools 56 exiting the virtual channels increases markedly when said ring shaped balloon 54 is inflated.

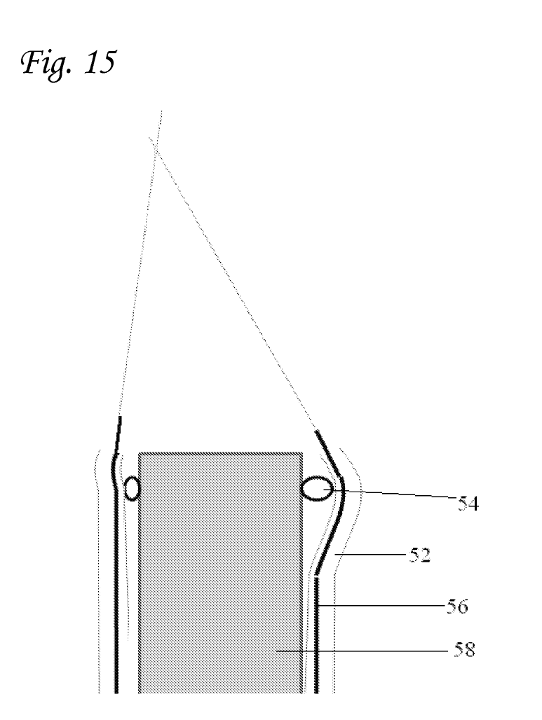

In addition, the presence of these additional balloons may also be advantageously used to control the angular direction of the virtual channel exit port. In this way, it is possible to move the virtual focus point of the tools contained within two or more virtual channels. Thus, in the device illustrated in FIG. 14, a ring shaped balloon 54 is located proximal to the distal end of the two virtual channels 52, at which height the distal portion of said channels curves inwards. Further distal advancement of the tools 56 contained within said channels would lead to their meeting at a focal point located a certain distance from the distal end of the device. However, it may readily be seen that it is possible to increase the distance of this focal point from the distal end of the device by increasing the degree of inflation of ring shaped balloon 54. A similar device is illustrated in FIG. 15 which differs from the device shown in FIG. 14 by virtue of the fact that the former device utilizes two discrete balloons 54 located just proximal to the distal exits of two virtual channels 52 (rather than the ring shaped balloon used in the other device). In this way, selective inflation of only one of the balloons may be used to change the exit angle of one of the tools 56, thereby creating a meeting place for the two tools that is biased away from the side of the inflated balloon.

It may thus be seen from the drawings discussed immediately hereinabove that the incorporation of the auxiliary balloons in the distal region of the virtual channels confers highly significant advantages on the present invention in terms of its ability to precisely direct the surgical tools that pass through said channels into the tissues that are situated beyond the distal end of the device.

It is to be noted that regardless of the specific balloon configuration actually used, the present invention makes provision for both the simultaneous inflation of each of the inflation channels, and also for the selective inflation of only some of said channels.

Fluid channels 3 may include jet-stream apertures of different dimensions and structures, different numbers and geometrical arrangement, and the apertures may cover the whole or part of the device (for example, only at the distal tip of the channel). Additionally, the device may include a plug or cap-like element 4. This element may be located at the proximal end of the device and may either be freely movable along the device or be provided as a separate element. The element 4 is inserted into the anus during activation of the device, to act like a plug and prevent leakage of fecal material from the colon. Element 4 may be rigid, semi-rigid, or elastic, and made to conform to the structure of the anus. In another embodiment of the invention, element 4 may be inflatable, using a fluid or gas. Additionally, the device comprises fluid (or gas) supply channels, leading from (and into) an external pumping mechanism (not shown in the illustrations) into (and from) the channels of the device. In the exemplary embodiment of the device shown in the figure, channel 5 connects the pump to inflatable channels 2 of the device, channel 6 connects the pump to plug element 4 of the device, and channel 7 connects the pump to fluid channels 3 of the device. In addition, the colonoscope distal end 8 is illustrated, exemplifying how the device of the invention may be fitted over the colonoscope, in a sleeve-like manner.

In one embodiment, the device may be designed, configured, and constructed, by starting with a single, unitary, tube, to which additional tubes are connected (for example by welding). To form apertures in fluid channel tubes 3, mechanical cutters or laser cutters may be used to remove selected material from the tube, until only the desired geometry, shape, and dimensions of the tube and the apertures remain.

There are several different approaches that may be employed in the manufacture of the device of the invention. One such exemplary approach comprises the steps of:
Step 1. Catheter tubes are manufactured by extrusion.
Step 2. Once a tube is formed, it will be cut to acquire the desired length and shape.
Step 3. Apertures are made at different areas of the selected tubes, as previously described.
Step 4. Connectors will be connected to the catheter proximal end, for example by ultrasonic welding.

Parts of the device can be manufactured, for example, by injection molding, which involves heating and injecting plastic material under pressure into a closed metal mould tool. The molten plastic cools & hardens into the shape inside the mould tool, which then opens to allow the moldings to be removed.

Referring again to FIG. 1, it should be noted that other devices may be inserted through the device of the invention and into the colon. These devices include (but are not limited to) grasping and cutting elements, in order to allow treatment of the colon, such as removal of colonic tissue for biopsy or cutting and removing colonic tumors, illumination devices (such a fiber optic devices) and cameras.

Exemplary dimensions of the device of the invention are as follows. The longitudinal dimension is in the range of about 20 cm to about 200 cm, preferably, about 150 cm. The diameter of the anchoring balloon elements is in the range of about 0.2 cm to about 1.5 cm, preferably, about 1 cm. The diameter of the fluid channel elements is in the range of between about 0.5 mm to about 5 mm, preferably, about 2 mm. The general thickness of the material used in the construction of the device is typically in the range of between about 0.01 mm (10 microns) and about 1.0 mm (1000 microns), preferably, about 0.5 mm (300 microns).

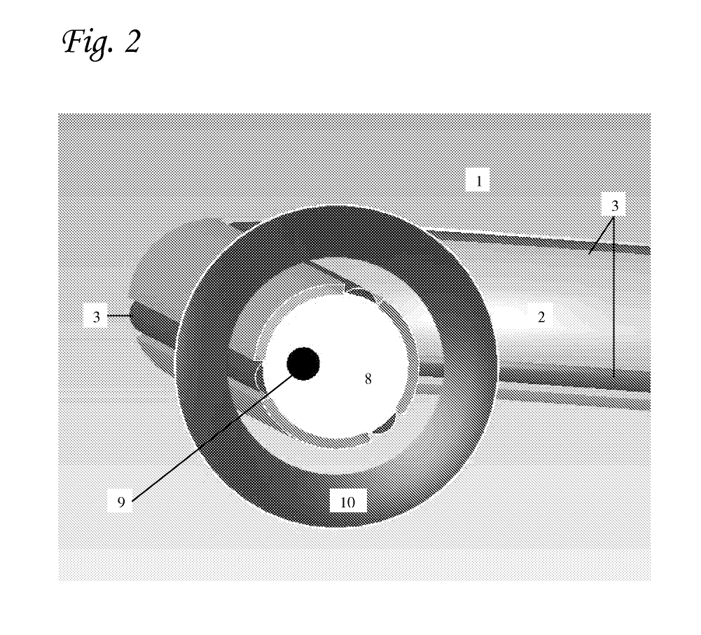

Referring again to the drawings, FIG. 2 is a schematic illustration of a cross section of an exemplary embodiment of the device 1 of the invention, fitted over a colonoscope in a deflated state within the colonic lumen 10. Anchoring balloon channels 2 are shown in their deflated state. Fluid channels 3 are also shown. In the example depicted in this figure, three anchoring balloon channels 2 and three fluid channels 3 are shown. It is to be recognized, however, that any convenient number (one or more) of either of these channels can be incorporated into the device. Additionally illustrated is a cross section of the colonoscope 8 and the colonoscope working channel 9.

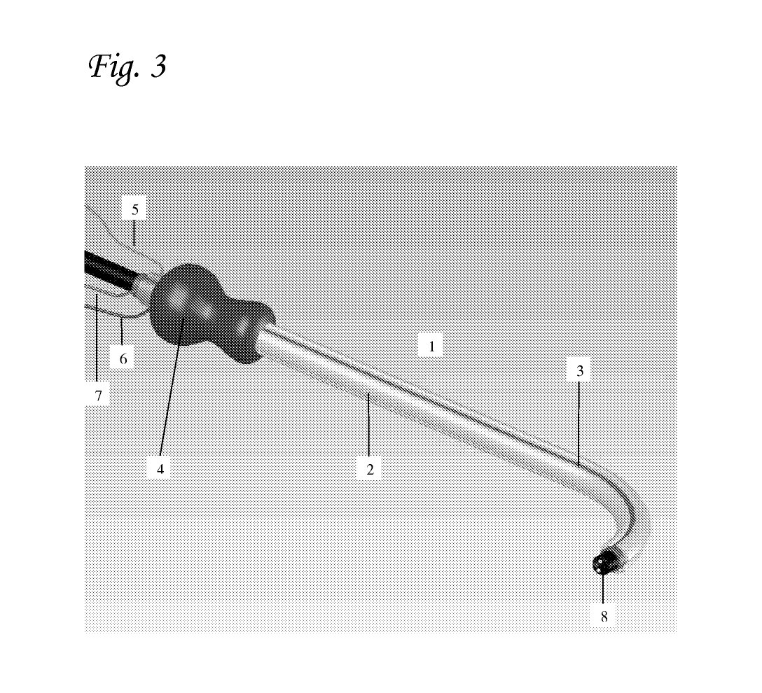

Referring again to the drawings, FIG. 3 is a schematic illustration of an exemplary embodiment of the device 1 of the invention, fitted over a colonoscope in an inflated state. The illustration shows inflatable anchoring balloon channels 2 in their inflated state as well as fluid channels 3. Additionally illustrated is an exemplary plug-like element 4. Also shown in the figure is channel 5, connecting the pump mechanism (not shown in the figure) to inflatable channels 2 of the device, channel 6 connecting the pump to plug element 4 of the device, and channel 7 connecting the pump to fluid channels 3 of the device. In addition, the colonoscope distal end 8 is illustrated, exemplifying how the device of the invention is fitted over the colonoscope, in a sleeve-like manner.

FIG. 4 is a schematic illustration of a cross section of an exemplary embodiment of the device 1 of the invention, fitted over a colonoscope in an inflated state, within the colonic lumen 10. Anchoring balloon channels 2 are shown in their inflated state, exemplifying the manner in which these inflated channels are capable of anchoring the device to the walls of the colon. Additionally, the figure also shows fluid channels 3. The number of channels shown in this figure—three anchoring balloon channels 2 and three fluid channels 3 is selected for illustration only and is not to be considered as limiting. Rather, any desired number of these channels can be incorporated into the device. When anchoring balloons 2 are in an inflated state, a series of “virtual channels” 15 are created, the medial boundary of said channels being formed by the fluid channels 3 (or the colonoscope itself, in a different embodiment wherein there are fewer fluid channels than anchoring channels), the superior and inferior borders by the anchoring channels 2, and the colonic wall laterally. In another preferred embodiment of the device the lateral wall is provided by an external membrane of the device (not shown but described in more detail hereinbelow) that is in contact with the colonic wall. Virtual channel 15 leads all the way from the distal end of the colonoscope to the proximal end (i.e. the end which during use is situated in the anal region). A suction force may be applied to the proximal end of the virtual channel (for example, by a suction applied via the pumping mechanism through plug element 4), thereby permitting aspiration of solid, semi-solid and liquid material from the distal region. In this way, fecal debris may be removed from the colon through the broad virtual channels 15, thereby cleansing the colon. During application of suction, the collapse of the colonic walls is prevented by the inflated balloon anchoring channels 2. In another preferred embodiment of the device, a membrane exists (not shown in the figure) between every two anchoring channels 2, such that in an inflated state, the membrane form a “roof” over channel 15, thereby preventing collapse of the colon wall during suction, and thus assisting in removal of fecal debris. Said membrane may be made of a polymer, an elastic material or a mesh, and may be solid or may have multiple apertures. In another preferred embodiment of the device, the internal or external or both surfaces of the device or the optional membrane (between channels 2) of the device are covered by a lubricating material. Lubrication of the internal surface may assist in the process of fitting the device over the colonoscope. Lubrication of the external surface and the optional membrane of the device may also assist in removing fecal material from the colon, thus cleansing the colon. In another preferred embodiment of the device, a means for spraying or pumping lubricating fluid is added to the device. Additionally illustrated in FIG. 4 is a cross section of the colonoscope 8 including the colonoscope working channel 9.

Referring again to the drawings, FIG. 5 is a schematic illustration of the distal head of an exemplary embodiment of the device 1 of the invention, fitted over a colonoscope 8, wherein the anchoring balloon channels 2 are shown in their inflated state. Fluid channels 3 are shown, as are the virtual channel 15, formed between the inflated anchoring channels 2 and the fluid channels 3, as explained hereinabove. In the non-limiting exemplary embodiment shown in the figure, the fluid channels 3 are mutually connected at the distal extremity of the device, thereby forming a ring-like structure, wherein said structure comprises a plurality of apertures 2 from which fluid jets may be expelled into the colon, in order to immobilize, materialize and break any fecal material in the colon. In other embodiments of the device, the fluid channels 3 may remain separate (i.e. not connected) at the distal end of the device, or alternatively, connected in another region of the device, or at multiple regions of the device. Apertures (side holes) 20 are shown at the distal part of the device. This is to illustrate that the device of the invention may have one or more apertures, preferably multiple apertures, on its external surface. Fluid may flow out of these apertures, and serve for both applying force on the colonic lumen—thereby assisting in preventing contact between the colonoscope and the colonic wall, and thus reducing friction between the colonoscope and the colonic wall, and also for dematerializing fecal matter. One preferred embodiment of the device may include side holes in the proximal part of the device and/or all along the colonoscope and/or in specified multiple locations along the colonoscope. Fluid apertures 20 and the fluid jets emitted therefrom may have different numbers, sizes and shapes, in order to cause different spray effects, thus maximizing their ability to break different types of material within the colon. The figure also depicts the distal head of the colonoscope 8 as well as the colonoscope working channel 9. In yet another embodiment of the invention, a pressure gauge is attached at the distal part of the device (not shown), for example in proximity to apertures 2, and may be used to measure intra-luminal pressure within the colon. This ability to monitor pressure at the distal tip of the colonoscope has the advantage of preventing the build-up of high fluid pressure within the device, which may otherwise lead to colonic perforation. It may thus be appreciated that this pressure monitoring capability increases the safety of the procedure for the patient. Pressure monitoring may be performed throughout the colonoscopy procedure, to increase its safety, or may be performed only during colon cleansing with the device of the invention.

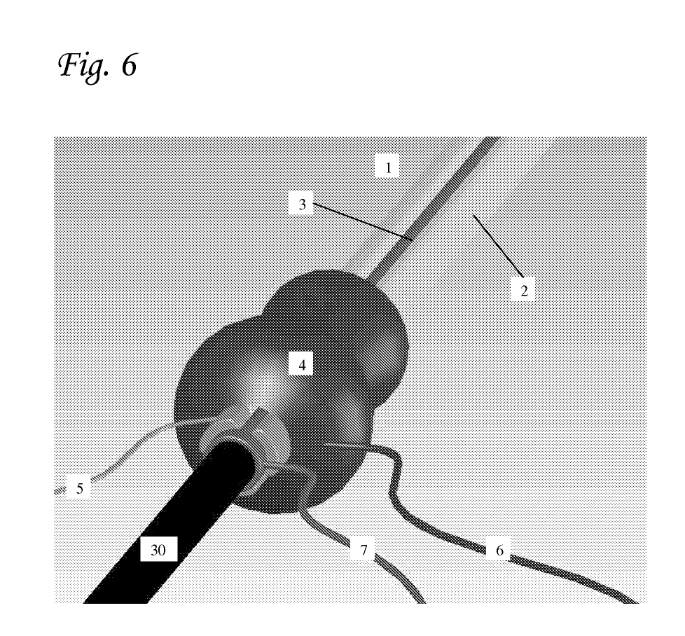

Referring again to the drawings, FIG. 6 is a schematic illustration of the proximal end of an exemplary embodiment of the device 1 of the invention, fitted over a colonoscope. Plug element 4 is illustrated, with channel 6 connecting it to an external pump (not shown in the figure). Channel 6 may transmit a suction force, which is transmitted through plug element 4 into channels 15 (not shown here, but shown in previous figures), which are formed when channels 2 of the device are inflated. Thus fecal material can be aspirated out of the colon, thereby cleansing the colonic lumen without the need to remove the colonoscope. Channels 5 are shown, connecting channel 2 to an external pump (not shown), thereby allowing inflation and deflation of said channels 2 with an appropriate expansion fluid (such as saline). Channels 7 are shown, connecting fluid channels 3 to an external pump (not shown), thus allowing pumping of fluid into said channels 3, which will be used in the colon to spray the fecal debris, dematerialize and break it, and assist with its removal. Additionally, the proximal part of the colonoscope 30 is shown, in order to exemplify the manner in which the device 1 is fitted in a sleeve like manner over the colonoscope.

The virtual channels (shown as 15 in FIG. 4) and the workflow described may be further used for all endoscope procedures wherein the operator would want to insert a clinical tool (therapeutic or diagnostic) through/along a shaft. Examples of this use of the virtual channels include (but are not limited to) the insertion of a device trough a virtual lumen along the guiding catheter in an intravascular procedure, the insertion of a tool into the stomach in an upper GI procedure, gastric lavage, and dilation of a urinary system stricture while at the same time allowing insertion of clinical tools and maintaining urinary flow.

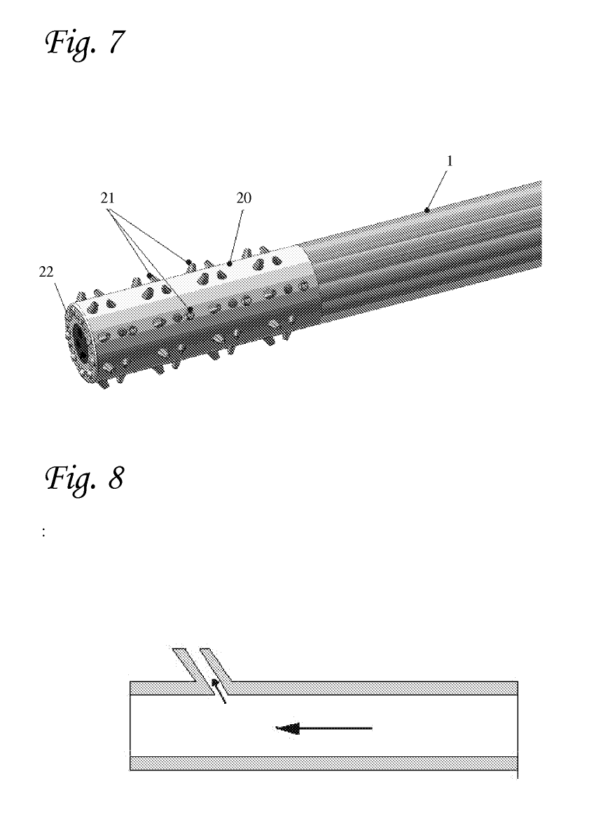

Referring again to the drawings, FIG. 7 is a schematic illustration of the distal head of the device 1, showing multiple exemplary fluid nozzles 20. By using different apertures/nozzles arrangement 21, 22 the fluid jets may create different configurations of flow: regular spray, regular pulsating and combined spray/pulsating. The distal part may be configured to scatter the fluid radially, radially with an angle and/or forward/backwards. Utilizing the nozzles may permit increase and focus of the flow from each aperture, due to normalization of the flow pattern, and may thus increase the efficacy of cleansing by the device.

It is known that rapid changes in lumen diameter may lead to turbulent flow, for instance when a fluid passes from the larger diameter lumen to the narrower nozzle, as shown in FIG. 8. Using the Turbulence principal, the invention combines variable ratio between water flow and optimal cleansing operation. Furthermore, the surface area of jet nozzles on the distal head corresponds to the overall area of impact of the fluids jets influencing the cleaning operation.

### EXAMPLE

### Typical Device of the Present Invention

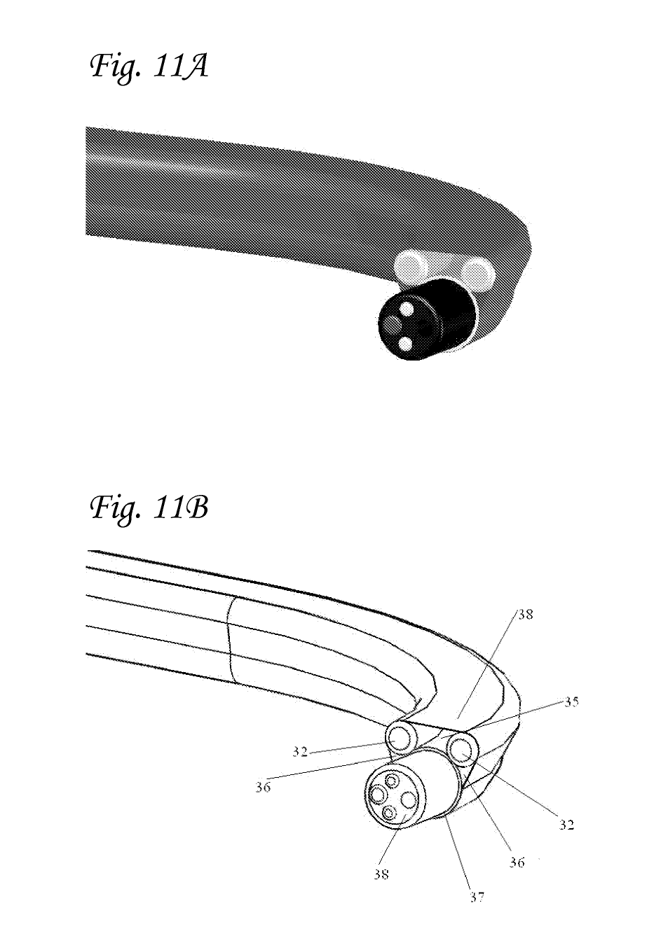

FIG. 11A is a three dimensional solid view of a typical preferred embodiment of the device of the present invention, comprising two longitudinal balloons, each having a diameter of 6 mm in their inflated state. When the balloons are inflated, three longitudinal lumens (“virtual lumens”; one large, two small) are created between the thin compliant internal sleeve upon which the balloons are mounted and the outer membrane of the device, which is brought into contact with the colonic wall by pressure from said inflated balloons. The various components of this device are shown in more detail in FIG. 11B, wherein the two inflatable balloons 32, are shown mounted on a compliant inner sleeve 37 and covered by external membrane 38. As illustrated, said inner sleeve 37 fits snugly over the colonoscope 38.

In this example, upon inflation of the balloons (inflatable channels) 32, three longitudinally-disposed virtual lumens are created: a large lumen 35 having a cross-sectional area of 30 mm² and two smaller lumens 36, each having a cross-section area of 6.5 mm². Each of these three lumens may be used for evacuation of debris and/or for irrigation (i.e. by means of pumping irrigation fluid such as saline therethrough). In addition, at different stages of the procedure, the lumens may also be used as working channels for the passage of instrumentation, as described hereinabove. The larger lumen is particularly advantageous in this regard, in view of its large surface area and volume, which permits the passage therethrough of larger instruments than would be possible through the working channel of the colonoscope itself.

In this typical working example, the inflatable channels (balloons) 32 are constructed from silicon rubber while the external membrane 38 is formed from Nylon 66 and the inner sleeve 37 from silicon rubber.

While the invention has been described in conjunction with specific embodiments and examples thereof, it is evident that many alternatives, modifications and variations will be apparent to those skilled in the art. Accordingly, it is intended to embrace all such alternatives, modifications and variations that fall within its spirit and broad scope.

1. A sleeve device comprising a thin-walled elongate internal tubular membrane which is suitable for being fitted around the external surface of an elongate medical instrument,
wherein at least one longitudinally-disposed inflatable channel having a closed distal end is affixed to the outer surface of said membrane,
and wherein each of said inflatable channels is capable of being expanded laterally such that an elongate medical instrument fitted with said sleeve and inserted into a body cavity would become anchored within said cavity, and such that the walls of said inflatable channel(s) would define the boundaries of one or more additional longitudinally-disposed large volume channels.

2. The sleeve device according to claim 1, wherein the large volume channels created upon inflation within a body cavity are capable of substantially retaining their initial volume and cross-sectional area upon the application of suction pressures of up to −760 mmHg to the proximal ends thereof.

3. The device according to claim 1, further comprising one or more open-ended longitudinally disposed non-inflatable channels affixed to the tubular membrane, wherein said channels are capable of allowing the passage of fluids along their length.

4. The device according to claim 1, further comprising an elongate longitudinally-disposed external tubular membrane enclosing all of the other elements of said device within its lumen.

5. The device according to claim 1, wherein the internal tubular membrane of said device is suitable for being fitted around the external surface of an endoscope.

6. The device according to claim 1, wherein the proximal end thereof is provided for means of connecting the open proximal ends of the various channels with a suction pressure source and a fluid reservoir.

7. The device according to claim 1, wherein each inflatable channel comprises a single expandable element.

8. The device according to claim 1, wherein each inflatable channel comprises a plurality of separate segments, wherein each segment is in fluid communication with its neighbor by means of a narrow opening.

9. The device according to claim 1, wherein each inflatable channel comprises a plurality of separate segments, wherein each segment is isolated from its neighbor and may be independently inflated and deflated by means of a separate inflation/deflation line attached thereto.

10. The device according to claim 1, further comprising an expandable balloon located at the distal end of said device, such that upon inflation thereof, balloon blocks the distal openings of the various channels.

11. The device according to claim 1, further comprising one or more circumferentially located balloons each of which is affixed to the external surface of the device in an annular manner, such that upon inflation thereof, the contents of the large volume channels are compressed.

12. The device according to claim 1, further comprising one or more auxiliary balloons attached to the outer surface of the internal membrane close to the distal end of said device.

13. The device according to claim 1, further comprising a proximal plug fitted in an annular manner around the external surface of said device, wherein said plug is adapted to form a fluid-tight seal when placed in the anal opening.

14. The device according to claim 1, further comprising pressure sensor elements located in various regions of said device, wherein said sensor elements are capable of detecting and reporting the fluid pressure levels within said regions.

15. A system for use in cleansing a body cavity comprising:
a device according to claim 1, and
a proximal control console.

16. The system according to claim 15, further comprising:
an extracorporeal pumping device capable of causing flow of cleaning fluids through one or more lumens of said device; and
an extracorporeal suction pressure source capable of causing aspiration of fluid, solid and semi-solid material through the large volume lumens of said device.

17. The system according to claim 15, further comprising a separate source of fluid for expanding the inflatable channels and/or other expandable elements of the device and a pumping device for delivering said fluid to said inflatable channels and/or other expandable elements.

18. The system according to claim 15, wherein the proximal control console comprises:
a display unit;
means for controlling a pumping device;
means for controlling a suction pressure source; and appropriate software for operating and co-coordinating the passage of cleaning fluid and expansion fluid into the device, and the application of suction pressure thereto.

19. A method for cleansing a body cavity comprising the steps of:
a) fitting a sleeve device around the external surface of an elongate medical instrument, wherein said device comprises a thin-walled elongate internal tubular membrane to the outer surface of which is fixed one or more inflatable channels, and optionally comprises one or more non-inflatable fluid-supply channels;
b) insertion of said elongate medical instrument together with the fitted sleeve into a body cavity, and advancement of said instrument until the distal end thereof is situated in the desired region;
c) inflation of said inflatable channels, such that said medical instrument becomes anchored within the desired region of the body cavity, and such that longitudinally-disposed large-volume virtual channels are created between said inflatable channels;
d) introduction of cleaning fluid into either the large-volume virtual channels or the non-inflatable fluid-supply channels, thereby causing the creating of fluid spray jets through openings located in either said virtual channels or in said fluid-supply channels, such that said fluid spray jets are directed outwardly towards debris located in said body cavity; and
e) performing suction of fluid, solid and semi-solid debris and other material from the body cavity;
f) deflation of said inflatable channels; and
g) optionally, movement of said medical instrument to another location within the body cavity to be cleansed;
wherein steps (b) to (g) do not adversely affect the ability of the operator to operate said medical instrument in the normal manner.

20. The method according to claim 19, wherein the body cavity to be cleansed is the colon, and the wherein the elongate medical instrument is a colonoscope.

21. A method for delivering endoscopic tools and accessories to a desired surgical site and for controlling the location of said tools and accessories at said site, comprising the steps of:
a) fitting a sleeve device around the external surface of an elongate medical instrument, wherein said device comprises a thin-walled elongate internal tubular membrane to the outer surface of which is fixed one or more inflatable channels, and optionally comprises one or more auxiliary balloons located on the outer surface of said membrane proximate to the distal end of said membrane;
b) insertion of said elongate medical instrument together with the fitted sleeve into a body cavity, and advancement of said instrument until the distal end thereof is situated close to the desired operating site;
c) inflation of said inflatable channels, such that said medical instrument becomes anchored within the desired region of the body cavity, and such that longitudinally-disposed large-volume virtual channels are created between said inflatable channels;
d) insertion of said endoscopic tools and/or accessories into one or more of said longitudinally-disposed large-volume virtual channels, and the distal advancement of said tools and accessories through said channels until said tools become located at the desired operating site; and
e) optionally inflating one or more of said auxiliary balloons in order to control the angle of exit of one or more of said tools from said virtual channels and/or the separation distance between multiple tools;
wherein steps (d) and (e) may be performed either simultaneously or sequentially, in either order.

22. The method according to claim 21, wherein said method forms part of a natural orifice transluminal endoscopic surgery (NOTES) procedure.
